(19) United States
(12) Patent Application Publication
(10) Pub. No.: US 2004/0099523 A1
(43) Pub. Date: May 27, 2004

(54) Catalytic simulation using radio frequency waves

(75) Inventor: Thomas J. Mohr, Maple (CA)

Correspondence Address: PARKHURST & WENDEL, L.L.P., 1421 PRINCE STREET, SUITE 210, ALEXANDRIA, VA, 22314-2805, US

(73) Assignee: OTEC RESEARCH INC., WOODBRIDGE (CA)

(21) Appl. No.: 10/670,238

(22) Filed: Sep. 26, 2003

Related U.S. Application Data
(63) Continuation of application No. 09/679,371, filed on Oct. 5, 2000, now abandoned.
Application No. 09/679,371 is a continuation-in-part of application No. 09/412,359, filed on Oct. 5, 1999, now Pat. No. 6,217,712.
Application No. 09/412,359 is a continuation-in-part of application No. 08/760,342, filed on Dec. 4, 1996, now abandoned.

(51) Int. Cl. C07C 5/00; H04H 1/00

(52) U.S. Cl. 204/157.15; 455/899

(57) ABSTRACT

The invention relates to a method of using radio frequency waves to artificially create catalytic action in a catalyst-free chemical reaction within a substance. To mimic or imitate the catalyst, radio frequency waves are transmitted through the substance at a signal strength sufficient to electronically reproduce the effect of the physical presence of a selected catalyst. The radio frequency waves have a selected transmission frequency substantially equal to a catalyst signal frequency of the selected catalyst, defined as the signal frequency determined by nuclear magnetic resonance of the selected catalyst. It is commonplace to use nuclear magnetic resonance to identify elements within a substance and the signal frequencies of various elements (including catalysts) are listed in widely published tables. To date, the mechanism by which catalysts bring about chemical reactions has been unknown. The inventor has recognised that the physical presence of a catalyst brings about a chemical reaction due to the emission of low intensity radio frequency waves from the catalyst with the signal frequency that is emitted being the signal frequency of the catalyst that is commonly determined by nuclear magnetic resonance. Therefore, the invention can be used to eliminate the need for expensive metallic catalysts, such as platinum. The invention electronically reproduces the effect of the physical presence of a catalyst by transmission of a radio frequency wave with a signal frequency equal to that signal frequency emitted by the catalyst and as determined by nuclear magnetic resonance of the catalyst.

### REFERENCE TO RELATED APPLICATIONS

This application is a Continuation-in-Part application of Ser. No. 09/412,359 filed Oct. 5, 1999 entitled: “Catalytic Simulation Using Radio Frequency Waves”, which is a Continuation-in-Part application of Ser. No. 08/760,342 filed Dec. 4, 1996 entitled: “Catalytic Resonance Simulator”, also invented by Thomas J. Mohr.

### TECHNICAL FIELD

The invention relates to a method of using radio frequency waves to artificially create catalytic action, in a catalyst-fee chemical reaction, to mimic or imitate the physical presence of a catalyst, by transmitting radio frequency waves through the substance at a signal strength sufficient to electronically reproduce the effect of the physical presence of a selected catalyst. The invention also relates to unique products yielded by this method and the uses thereof

### BACKGROUND OF THE INVENTION

Catalysts are used in many conventional chemical reactions frequently in the form of precious metals plated on metal anodes or cathodes, ceramic catalytic converter structures and the like. The catalysts are inert and are not consumed during the chemical reaction but their physical presence is required to carry out the reaction or enhance effectiveness. The actual mechanism by which a catalyst exerts influence on the chemical reaction is not known; however, the effect of the presence of various catalysts has been well established.

The disadvantage of using conventional catalysts is that the initial cost of such precious metals is very high, periodic cleaning or replacement is required to ensure maximum efficiency, and disposal of materials coated with catalysts involve catalyst reclaiming, high cost and possible heavy metal contamination. Minimal amounts of catalyst are used as a result of their high cost. The effectiveness of a catalyst is not controllable and the conventional manner of increasing the effect of a catalyst is to increase exposure to the catalyst during the chemical reaction.

It is an object of the present invention to provide a method which eliminates, or at least reduces, the physical use of catalytic materials in a chemical reaction, thereby minimizing the cost of conducting the chemical reaction as well as obviating other disadvantages of conventional catalyst use.

Further objects of the invention will be apparent from review of the disclosure and description of the invention below.

### DISCLOSURE OF THE INVENTION

The invention relates to a method of using radio frequency waves to artificially create catalytic action in a catalyst-free chemical reaction. To mimic or imitate the catalyst, radio frequency waves are transmitted through the reaction mixture at a signal strength sufficient to electronically reproduce the effect of a selected catalyst. The radio frequency waves have a selected transmission frequency that is substantially equal to the signal frequency of the selected catalyst as determined by nuclear magnetic resonance.

It is commonplace to use nuclear magnetic resonance to identify elements within a substance and the signal frequencies of various elements (including catalysts) are listed in widely published tables.

To date, however, the exact mechanism by which catalysts bring about chemical reactions has been unknowns There is no doubt that “inert” catalysts are essential for some chemical reactions to occur and can clearly optimize other chemical reactions, even though the catalyst is not consumed or altered during such reactions.

The inventor has recognized that the physical presence of a catalyst brings about a chemical reaction due to the emission of low intensity radio frequency waves from the catalyst. These emitted radio waves have a very low signal strength with a signal frequency equal to the signal frequency of the catalyst that is commonly determined by nuclear magnetic resonance and conventionally used to identify the catalyst.

Therefore, the invention relates to the use of electronically generated radio frequency signals to imitate catalysts and eliminate the need for the actual physical presence expensive metallic catalysts, such as platinum. The invention electronically reproduces the effect of the physical presence of a catalyst by transmission of an artificially produced radio frequency wave signal with a signal frequency equal to the natural signal frequency emitted by the catalyst as determined by nuclear magnetic resonance.

In another aspect, the present invention relates to the application of this novel method of catalyzing a reaction to produce a unique form of super-oxygenated water comprising dissolved oxygen in stabilized single atom form at a concentration of greater than 9.5 milligrams per litre. Such super-oxygenated water is particularly useful for the manufacture of various medicinal solutions including solutions for intravenous, topical or oral application.

By providing an electronically simulated radio frequency transmission which mimics the radio frequency transmission resulting from the physical presence of a selected catalyst, the use of an actual catalyst can often be completely eliminated. In some applications, it may be preferable to retain the presence of a conventional catalyst but to enhance its effect by adding a simulated radio frequency transmission of strength or amplitude equal to or greater than the catalyst's own transmission. In this way, the cost of conducting a particular catalyzed chemical reaction is at least reduced by minimizing or eliminating the use of expensive precious metal catalysts. The cost of reclaiming and repairing the catalytic converter used in the reaction can also be reduced by use of the present method.

Although the drawings and related description of the invention concern preferred embodiments, it will be understood that the invention in it's broad scope includes application to any reaction in which the presence of a simulated catalyst is of benefit.

Further details of the invention and its advantages will be apparent from the detailed description and drawing included below.

### BRIEF DESCRIPTION OF THE DRAWINGS

In order that the invention may be readily understood, two preferred embodiments of devices which operate using the method of the invention and variations thereof will be described by way of example, with reference to the accompanying drawings wherein:

FIG. 1 is a schematic view of one embodiment of a device utilizing the method of the invention applied to a liquid electrolyte reaction within a conducting pipeline housing an electrolytic cell, a submerged standing wave antennae and a radio frequency generator,

FIG. 2 is a partially longitudinal sectional detail view of the electrolytic cell and antennae housing;

FIG. 3 is a sectional transverse view through the antennae housing showing the standing wave antennae and oscillator coil;

FIG. 4 is a schematic view of a second embodiment also using the method of the invention applied to a liquid electrolytic reaction within a cylindrical reaction chamber, and

FIG. 5 is a chat illustrating the % increase in blood oxygen levels on ingestion of super-oxygenated water prepared using the method of the present invention.

### DETAILED DESCRIPTION OF PREFERRED EMBODIMENTS

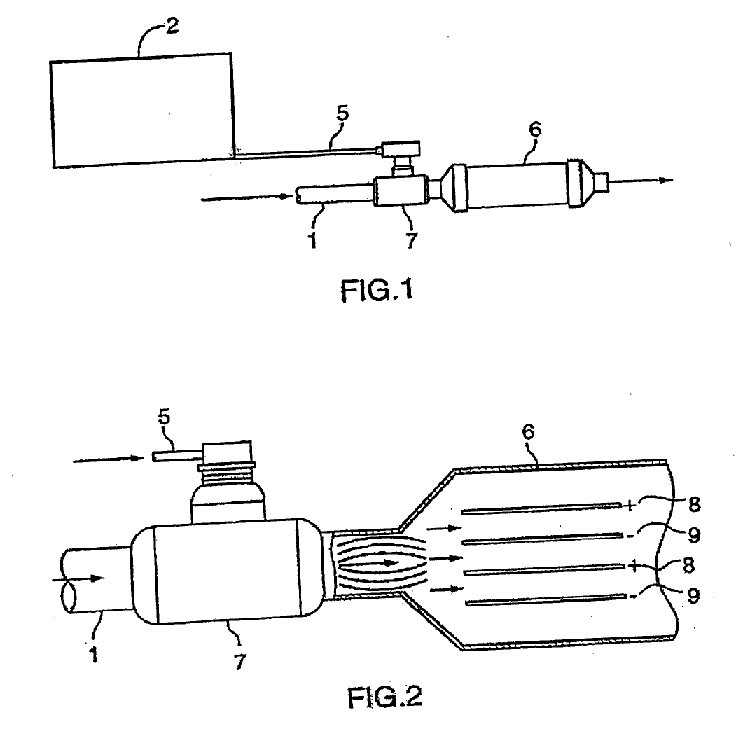

FIG. 1 illustrates a device which can be used to carry out the method of the invention. In this example, the method of the invention is used to artificially create catalytic action in a catalyst-free chemical reaction that is conducted through a pipeline in the direction of the arrows shown Radio frequency waves are used to artificially create this catalytic action as liquid electrolyte is conveyed through a pipeline 1 in the direction of arrows by a pump (not shown). Electrolysis is the reaction illustrated in this example; however, it would be understood that any reaction requiring catalysis could benefit from the present catalytic method. Therefore, any means for containing a substance during a chemical reaction may be used including agitator tanks, cracking towers, settling tanks, etc.

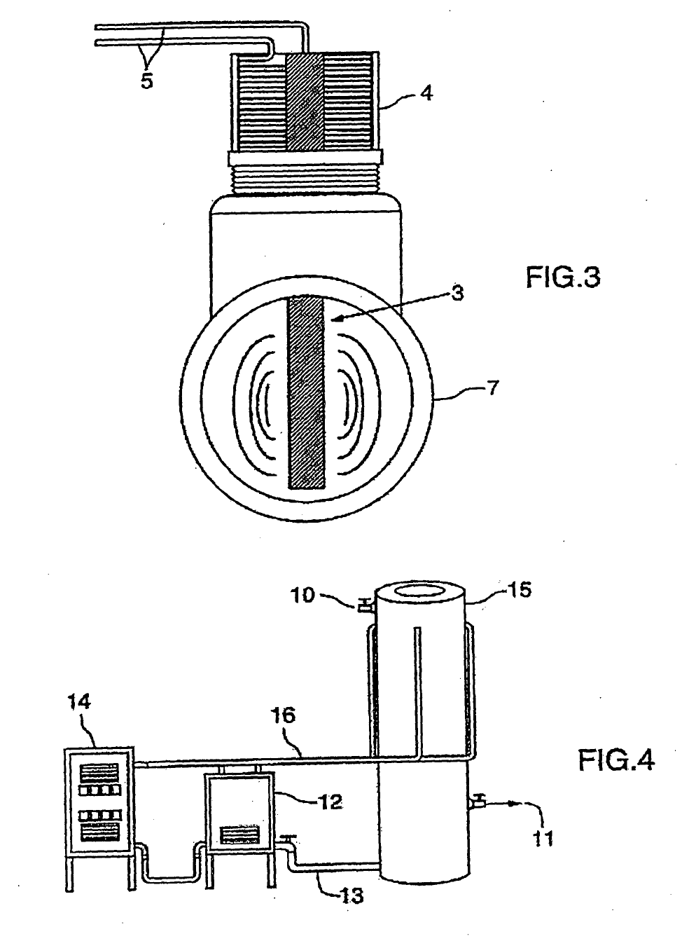

A radio frequency wave transmitter 2 is disposed in communication with the electrolyte by means of a standing wave antenna 3 which is submerged in the electrolyte within the pipe 1 and the section 7. As best shown in FIGS. 2 and 3, the antenna 3 communicates with an oscillator coil 4 which receives the radio frequency signal from the frequency generator 2 via a cable 5.

In the embodiment illustrated in FIGS. 1, 2, and 3, the chemical reaction is electrolysis carried out within an electrolytic cell 6. Within the cell 6 is disposed cathode 9 and anodes 8 both connected to a source of an electric current and immersed in the electrolytic substance. Preferably the electrolyte is water and the chemical reaction comprises electrolysis to increase the dissolved oxygen gas within the water as it passes through the electrolytic cell 6.

In operation, the method involves transmitting radio frequency waves generated from the frequency generator 2 and emitted via the standing wave antenna 3 to conduct the radio frequency waves through the electrolyte at a signal strength sufficient to electronically reproduce the effects of the physical presence of a catalyst

In a traditional electrolysis reaction, the cathodes 9 and anodes 8 would be plated with a metallic catalyst such as platinum. However, in the method of the present invention, the cathodes 9 and anodes 8 are not plated with a catalyst since the radio frequency waves transmitted via the antenna 3 artificially create the same catalytic action. The radio frequency waves have a selected transmission frequency substantially equal to the signal frequency of the selected catalyst. This catalyst signal frequency is defined as the signal frequency conventionally determined by nuclear magnetic resonance of the selected catalyst. For example, where the selected catalyst is platinum, the radio frequency transmission is in the order of 929 megahertz. This frequency and the frequencies of other catalysts are commonly listed in NMR tables and are readily available to those skilled in the relevant art (for example, in the well-known and widely available Encyclopedia of Chemistry and Physics textbook).

It will be understood, however, that nuclear magnetic resonance is conventionally used to identify elements that are present within an unknown substance. The elements under bombardment of external radio frequencies in the presence of a strong magnetic field emit a signal radio frequency which can be detected and which definitely identifies the element. These signal frequencies are listed in NMR tables and are used by those skilled in the art to conclusively identify the elements that are present within an unknown substance.

In contrast, the present invention utilizes these established radio frequencies to electronically artificially create catalytic action in a catalyst free chemical reaction. Therefore, it will be understood that the method does not use conventional nuclear magnetic resonance but rather uses the result of NMR to determine the appropriate radio frequency to be used and twitted through the antenna 3.

For example, if the catalyst which is necessary for a chemical reaction is uranium, iridium or ruthenium, the radio frequency transmitted from the frequency generator 2 and emitted by the antenna 3 is adjusted accordingly to match the radio frequency of Uranium, iridium or ruthenium from NMR tables.

By experimentation, it has been concluded that the precise radio frequency to be transmitted through the substance may require slight variation to optimize the reaction depending on the size and shape of the reaction chamber and other details of the physical containment of the chemical reaction.

The radio frequency generator 2 can be used to fine-tune the transmission frequency while monitoring the reaction to optimize the reaction. For example, in the device shown in FIG. 1, the generation of oxygen gas within the water electrolyte can be monitored easily by using a dissolved oxygen meter downs of the electrolytic cell 6. In addition, the method can include fine tuning the signal strength emitted by the frequency generator 2 while monitoring the reaction with the dissolved oxygen meter to optimize the reaction. Many chemical reactions also require the regulation of temperature to optimize the reaction and this step can be accomplished by conventional means such as chillers or heaters for the electrolyte.

FIG. 4 shows an alternative version wherein a reaction tower 15 is utilized for the electrolysis reaction. In this second device, utilizing the method of the invention, fresh liquid electrolyte enters inlet 10 and reacted electrolyte is removed via outlet 11. A chiller unit 12 maintains optimum electrolyte temperature for the reaction as electrolyte is removed via the base pipe 13, proceeds through the chiller 12 and then through the process unit 14. The process unit 14 houses the frequency generator 2, electrolytic cells 6, standing wave antenna 6 and all related components in a single compact unit. Reacted electrolyte is returned to the tower 15 via return pipe 16. Gases and other reaction products may be removed within the tower 15 and reacted electrolyte removed for further processing via outlet 11.

To recap therefore, the method of the invention creates catalytic action within a chemical reaction by means of induced radio frequency energy to replace the metallic catalyst. The conventional method of producing hydrogen gas from water in an electrolytic process is to pass the water rough an electrolytic cell comprising a titanium anode and cathode plated with platinum as the catalyst. As water flows through the electrolytic cell, a stream of electrons in the form of direct electrical current pass through the water between the cathode and anode. With the platinum present in the electrolytic cell, the reaction causes a breakdown in the bond between the hydrogen and oxygen atoms. The hydrogen atoms are liberated as H2 gas at the cathode and are released into the atmosphere or collected after exiting the electrolytic cell. The oxygen atoms that are sea from the hydrogen in the water molecules then collect at the anode and are carried off by the water as dissolved oxygen gas. If the platinum catalyst is not present in the electrolytic cell, this reaction will not be complete and no dissolved oxygen can be measured in the exit stream. As well, no hydrogen gas is measured exiting from the outlet stream. In a conventional reaction, therefore, the platinum catalyst is absolutely essential for production of these gases.

In contrast, the invention provides a method wherein an electrolysis reaction is conducted in a similar electrolytic cell with the important difference that no platinum plating is provided on either electrode. Conventional wisdom would dictate that the electrolytic reaction could not occur in such circumstances. However, when a radio frequency energy is provided in the electrolyte cell in the range of 9.29 megahertz, dissolved oxygen is in fact measured by a submerged dissolved oxygen probe in the stream after it exits the electrolytic cell. The radio frequency of 9.29 megahertz matches the frequency signal of platinum as measured by conventional nuclear magnetic resonance and as conventionally listed in NMR tables. The introduction of this radio frequency into the reaction by means of a submerged antenna artificially creates catalytic action in an otherwise catalyst-free chemical reaction.

When no radio frequency signal is introduced into this electrolytic cell, very little measure of dissolved oxygen is present in the exit stream. When the radio signal is introduced and fined tuned for frequency and signal strength, the dissolved oxygen meter reads increasing levels of dissolved oxygen until an optimum frequency and amplitude is reached. The result of utilizing the method of the invention indicates that the presence of an appropriate radio frequency replaces the physical presence of any metallic catalyst in the reaction. Using the known signal frequency of 9.29 megahertz for platinum as determined by nuclear magnetic resonance, as the sa point for fine tuning an adjustable signal gives an immediate increase in the dissolved oxygen content of the exit stream. Further fine tuning of the frequency strength of the signal improves the production of dissolved oxygen until an optimum combination of frequency and signal strength is discovered by experimentation. Use of radio frequency, as opposed to physical presence of actual platinum within the electrolytic cell, reduces the cost of cell manufacture and creates higher dissolved oxygen content in the oxygenated water produced by the electrolytic cell.

Moreover, use of the present method, as outlined above, in the electrolysis of water results not only in high concentrations of dissolved oxygen in the water, but results in a unique form of dissolved oxygen in the water. Specifically, the dissolved oxygen is in the form of stabilized single atom oxygen rather than molecular oxygen (O₂) gas. The ability of the method to yield this stabilized oxygen atom in large concentrations is directly related to the electronic simulation of the catalyst which provides a means for a specific reaction to occur during the electrolysis. That is, when the oxygen atoms are separated from the hydrogen/oxygen bond that forms the water (H₂O) molecule, the separated oxygen atoms each accept an electron from the process that stabilizes their outer valence rings, thereby stabilizing the singular oxygen atoms and reducing their need to bind with another oxygen atom to form oxygen (O₂) molecules which exist primarily in gaseous form This renders the oxygenated water produced by the present method particularly useful for medicinal solutions, especially intravenous solutions. The absence of oxygen gas molecules in intravenous solutions minimizes the possibility of the formation of potentially fatal gas embolisms in the blood on administration of such solutions.

Accordingly, oxygenated water produced using the method of the present invention is particularly useful in the manufacture of medicinal solutions selected from the group consisting of: intravenous solution; electrolytic solution; saline solution; topical burn solution; topical skin treatment solution; oral rinse treatment solution; dental rinse treatment solution; ingestible blood oxygen content elevating solutions; ingestible blood oxygen partial pressure elevating solutions; bactericide; virus killing solution; anaerobic tumour treatment solution; physical injury immersion treatment solution; and brain tissue treatment solution.

In addition, the super-oxygenated water can be used as a disinfectant and preservative for many tissues or organic products which are subject to deterioration. The polysaccharide cell walls of bacterial organisms are oxidized when exposed to super-oxygenated water. As a result, bacteria exposed to such water is killed through oxidization. This method of killing bacteria is novel and has very wide application, especially in the medical field. For example, in the current transport of human transplant organs and tissue, these organs and tissue are usually packed in ice, which permit transport within a certain period of time before the organs and tissue deteriorate to the extent they cannot be used Super-oxygenated water can be frozen and used as packing to simultaneously cool the tissues, disinfect them and provide additional oxygen through osmosis to inhibit bacterial growth and slow the deterioration of the tissue and organs thereby increasing the transport time that tissue and organs for transplant can tolerate.

In living organisms as well, topical application of super-oxygenated water solutions can increase the delivery of oxygen to exposed surfaces through osmosis. External treatment of burns currently involves immersing burn tissues in medicated water which promotes healing. By immersing burned tissues in super-oxygenated water, harmful bacteria are killed as mentioned above, and in addition, rapid healing is enhanced by provision of additional oxygen to the affected area.

Blood oxygen levels can be increased well, by surface immersion of the body or parts of the body in a vessel containing super-oxygenated water since the oxygen dissolved in the super-oxygenated water passes through cell walls into the blood via osmosis. Skin disorders, injury to soft tissues, abrasions, spa and burns cam also be treated by immersion of the effected area or bating in super-oxygenated water, in addition to wrapping the skin area in bandages soaked in super-oxygenated water. Gum disease, oral infections and dental infections can be treated as well by topical application, or rinsing the mouth areas with super-Oxygenated water which disinfects and increases oxygen delivery to the adjacent areas.

Ingestion of super-oxygenated solutions increases the delivery of oxygen to the blood and cells, thereby increasing the blood oxygen content and elevating the partial pressure of oxygen in the blood. Intravenous injection of super-oxygenated water as a component of saline or Ringer's solutions likewise increases the blood oxygen content and elevates the partial pressure of oxygen in the blood, as well as enhancing the uptake of electrolytes, salts and minerals.

Many diseases and malfunctions of organs or deterioration of body tissues can be attributed to the lack of oxygen Such maladies can be treated either with ingestion or intravenous injection of super-oxygenated water containing high concentrations of oxygen. Such maladies include heart attack damaged tissues, certain brain disorders and problems associated with poor circulation, such as coronary diseases, arthritis, etc.

Certain maladies effect only specific areas of the body and these can be treated by targeting these specific areas with super-oxygenated water. For example, cells of anaerobic tumors are killed or have their rapid growth impeded through exposure to elevated oxygen concentrations delivered in the form of super-oxygenated water. Brain disorders and heart attack effected issues can also benefit from elevated oxygen concentrations on targeted exposure to super-oxygenated water solutions delivered either through ingestion or intravenous injection.

Medicinal solutions, such as those identified above, are readily made using conventional methods as would be familiar to those of skill in the art. Specifically, the methods used to make medicinal solutions in accordance with the present invention correspond to the usual method of their manufacture with the exception that the water used in such solutions is replaced with super-oxygenated water comprising stabilized dissolved oxygen atom at a concentration of greater than 9.5 milligrams per litre as produced by the novel catalytic method herein described. For example, saline solution is prepared in the usual manner with super-oxygenated water produced by the method of the present invention and having a concentration of NaCl of about 0.9%, or within an acceptable range thereof. Other medicinal solutions are similarly made with the super-oxygenated water, the amounts of all other components of the solutions remaining unchanged.

Embodiments of the present invention are exemplified in the following specific example which is not to be construed as limiting

### EXAMPLE

An experiment was conducted utilizing the method of the invention as follows. Water is pumped through the piping 1 and electrolytic cell 6. Direct current power supply is connected to the cathodes and anodes 9 and 8 and sends a stream of electrons through the water between the anode 8 and cathode 9 of the electrolytic cell 6. The frequency generator 2 supplies a selected frequency signal through the submerged antenna 3 upstream of the cell 6. The dissolved oxygen content of the exit stream from the cell is measured via a submerged probe connected to a dissolved oxygen meter. No platinum catalyst is present in the electrolytic cell 6. In contrast, a conventional electrolytic cell would include catalysts plated on the cathode. With the frequency generator off, the flow of input water is measured for dissolved oxygen content, typical results are in the order of 7 milligrams per litre (mg/l). Power is applied to the electrolytic cell 6 by DC power supply and the exit stream of water is measured again for dissolved oxygen content. A typical reading is only marginally higher in the order of 7.6 to 8.5 mg/l. However, when the frequency signal is applied at 9.29 megahertz and signal strength is set at 0.5 watts, the dissolved oxygen reading increases to the order of 14 mg/l.

Further increasing the signal strength to 1.05 watts and fine tuning the frequency of the signal in the order of 9.29 megahertz, increases the dissolved oxygen reading to over 20 mg/l typically.

When the frequency generator 2 is switched off, the dissolved oxygen reading reverts to the 7.6 to 8.5 mg/l range. Switching the frequency generator 2 back on raises the dissolved oxygen reading back up to the 20 mg/l range.

An additional means to create a frequency signal within an electrolytic cell (with the intent to simulate the frequency signal of a known catalyst in the reaction without physical presence of a metallic catalyst) is to oscillate the voltage of a DC power supply connected to the anode and cathode instead of providing a separate radio frequency.

In this second embodiment of the method the voltage is supplied from a DC power supply oscillated within a selectable range to create a sine wave frequency within the electric current and supply to the electrolytic cell anode and cathode. When the current travels from the positive electrode to the negative electrode through the liquid electrolyte, the voltage oscillation frequency can be adjusted to perform the function of the metallic catalyst normally plated onto one or both electrodes. The function of oscillating the voltage at a known frequency accomplishes the identical result as explained above in simulating the frequency signature of a catalyst. Therefore, instead of providing a separate standing wave antenna with variable frequency generator, the second embodiment merely oscillates the voltage slightly at a chosen frequency in a like manner.

Characteristics of the Super-Oxygenated Water

A number of tests were conducted to determine the characteristics of the oxygenated water produced using the method described herein and particularly in the foregoing example.

At the outset, the capacity of the water to maintain its oxygenated form while exposed to atmospheric pressure, i.e. in an open container, was determined. The following results were obtained using a CIBA Corning 278 Blood Gas System at 37° C. and at various time intervals following exposure of the water to atmospheric pressure, e.g. T=0 minutes (i.e. immediately upon opening the bottle), T=7 min and T=16 hours:

| | T = 0 | | T = 7 minutes | | T = 16 hours | |
|---|---|---|---|---|---|---|
| Acid/Base: | | | | | | |
| pH | 7.423 | | 7.420 | | 7.576 | |
| PCO₂ | 11.2 | mmHg | 11.5 | mmHg | 8.3 | mmHg |
| PO₂ | 529.2 | mmHg | 475.2 | mmHg | 315.0 | mmHg |
| HCO₃a | 73 | mmol/L | 7.4 | mmol/L | 7.7 | mmol/L |
| HCO₃s | 14.9 | mmol/L | 15.0 | mmol/L | 18.0 | mmol/L |
| CO₂ | 7.7 | mmol/L | 7.8 | mmol/L | 8.0 | mmol/L |
| BE(vt) | −12.6 | mmol/L | −12.5 | mmol/L | −8.2 | mmol/L |
| BE(vv) | −15.4 | mmol/L | −15.3 | mmol/L | −13.0 | mmol/L |
| Oxygen Status: | | | | | | |
| PO₂ | 529.2 | mmHg | 475.2 | mmHg | 315.0 | mmHg |
| O₂SAT | 99.9% | | 99.9% | | 99.8% | |

These results indicate that this oxygenated form of water is stable and maintains a high concentration of dissolved oxygen for an extended pod of time upon exposure to the atmosphere. These results also indicate that solutions made with this form of oxygenated water will have an extended shelf life. Moreover, the results indicate that the water is particularly suitable for use in medicinal solutions since its pH is very close to the physiological pH of 7.4.

The oxygenated water was also tested for the presence of potentially harmful oxygen free radicals as set out below.

1. Superoxide Radical—Analysis of the sample was conducted using the RANSOD (Superoxide dismutase) kit (Randox Laboratories Ltd). Superoxide radical was undetected.

2. Hydrogen Peroxide—Analysis of the sample was conducted spectrophotometrically based on the peroxidase-oxidase reaction (Eur. J. Biochem.2238:785;1996). Using this reaction, hydrogen peroxide was undetectable (lower than 10 m).

3. Hydroxyl Radical—The sample was analyzed by gas chromatography-mass spectrometry (GC/MS) using salicylate as a trapping agent (Clin. Biochem. 30:41; 1997). No hydroxyl radical (lower than 10 micromolar) was detected using this method.

4. Peroxides—An assay for peroxides and oxidizing substances was done using the starch iodide test and absorbance at 600 nm. Using this assay, there were no detectable peroxides in the sample (lower than 50 micromolar).

5. Reducing Substances—An assay for reducing substances was done using a test with dithionitrobenzoic acid and absorbance at 412 nm. There were no detectable reducing substances in the sample.

6. Organic Substances—An oxidation test using potassium permanganate showed no detectable levels of oxidizable organic compounds

These results provide evidence that the oxygenated water comprises a stabilized oxygen atom that presents no adverse effects on use.

Preliminary Clinical Testing

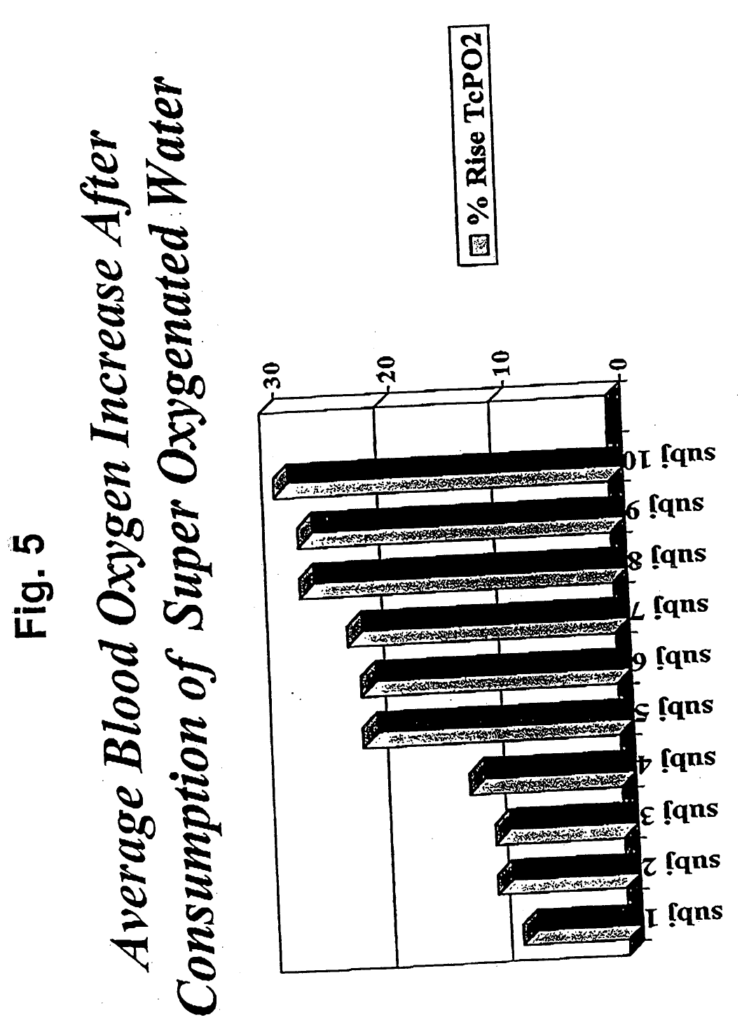

Preliminary clinical testing of the super-oxygenated water has been conducted to determine the effects on ingestion of the water. Ten volunteers were tested following ingestion of 10 ml/kg of super-oxygenated water prepared as described above. Approximately 10 minutes following ingestion, TcPO₂ (blood oxygen) was determined for each volunteer. The results for each volunteer are indicated in the chart shown in FIG. 5 and indicate that blood oxygen levels do increase on ingestion of the super-oxygenated water, the average maximal increase in TcPO₂ being 19.4% and the median maximal increase in TcPO₂ being 22%.

Although the above description and accompanying drawings relate to specific preferred embodiments as presently contemplated by the inventor, it will be understood ta the invention in its broad aspect includes equivalents of the method steps and elements described and illustrated.

We claim:

1. A method of using radio frequency waves to imitate the presence of a catalyst and artificially create catalytic action in a catalyst-free chemical reaction comprising:
transmitting radio frequency waves through the reaction mixture at a signal strength sufficient to electronically reproduce the effect of the physical presence of a selected catalyst,
wherein the radio frequency waves have a selected transmission frequency substantially equal to the signal frequency of said selected catalyst as determined by nuclear magnetic resonance.

2. A method according to claim 1 comprising:
fine tuning the transmission frequency while monitoring the reaction to optimize the reaction.

3. A method according to claim 1 comprising:
fine tuning the signal strength while monitoring the reaction to optimize the reaction.

4. A method according to claim 1 wherein the catalyst-free chemical reaction is electrolysis carried out within an electrolytic cell comprising a cathode and an anode each communicating with a source of electrical current and each immersed in an electrolyte substance.

5. A method according to claim 4 wherein the radio frequency waves are transmitted via a standing wave antennae submerged in the electrolyte.

6. A method according to claim 5 wherein the electrolytic cell is housed within a conduit and electrolyte is pumped through the conduit.

7. A method according to claim 6 further including the step of regulating the temperature of the electrolyte to optimize the reaction.

8. A method according to claim 6 wherein the electrolyte is water and the reaction comprises electrolysis to increase the dissolved oxygen gas within the water.

9. A method according to claim 8 fit including the step of monitoring the dissolved oxygen concentration of the water passed from the electrolytic cell.

10. A method according to claim 1 wherein the selected catalyst is selected from the group consisting of: platinum; rhenium; iridium; and ruthenium.

11. A method according to claim 10 wherein the selected catalyst is platinum and the transmission frequency is in the order of 929 megahertz.

12. A method of using radio frequency waves to imitate the presence of an inert metallic catalyst and artificially create catalytic action in a catalyst-free chemical reaction comprising:
transmitting radio frequency waves through the reaction mixture at a signal strength sufficient to electronically reproduce the effect of the physical presence of a selected inert metallic catalyst,
wherein the radio frequency waves have a selected transmission frequency substantially equal to the signal frequency of said selected inert metallic catalyst as determined by nuclear magnetic resonance.

13. The method of claim 12 comprising:
fine tuning the transmission frequency while monitoring the reaction to optimize the reaction.

14. The method according to claim 12 co g:
fine tuning the signal strength while monitoring the reaction to optimize the reaction.

15. The method according to claim 12, wherein the catalyst-free chemical Don is electrolysis carried out within an electrolytic cell comprising a cathode and an anode, each communicating with a source of electrical current and each immersed in an electrolyte substance.

16. The method according to claim 15, wherein the radio frequency waves are transmitted via a standing wave antennae submerged in the electrolyte.

17. The method according to clam 16, wherein the electrolytic cell is housed within a conduit and electrolyte is pumped through the conduit.

18. The method according to claim 17, further including the step of regulating the temperate of the electrolyte to optimize the reaction.

19. The method according to claim 17, wherein the electrolyte is water and the reaction comprises electrolysis to increase the dissolved oxygen gas within the water.

20. The method according to claim 19, further including the step of monitoring the dissolved oxygen concentration of the water passed from the electrolytic cell.

21. The method according to claim 12, wherein the selected catalyst is selected from the group consisting of platinum, rhenium, iridium, and ruthenium.

22. The method according to claim 21, wherein the selected catalyst is platinum and the transmission frequency is in the order of 929 megahertz.

23. Super-oxygenated water comprising stabilized dissolved oxygen atom at a concentration of greater than 9.5 milligrams per litre

24. A medicinal solution containing super-oxygenated water as defined in claim 23, said solution being selected from the group consisting of: intravenous solution; electrolytic solution; saline solution; topical burn solution; topical skin treatment solution; oral rise treatment solution; dental rinse treatment solution; ingestible blood oxygen content elevating solutions; ingestible blood oxygen partial pressure elevating solutions; bactericide; virus killing solution; anaerobic tumour treatment solution; physical injury immersion treatment solution; and brain tissue treatment solution.

25. A medicinal solution as defined in claim 24, wherein said solution is saline solution.

26. A preservative containing super-oxygenated water as defined in claim 23, said preservative being selected from the group consisting of fluid preservative; ice for use as a cooling preservative; live human organ preservative; and live human tissue preservative.

27. The use of super-oxygenated water as defined in claim 23 for the manufacture of a medicinal solution selected from the group consisting of: intravenous solution; electrolytic solution; saline solution; topical burn solution; topical skin treatment solution; oral rinse treatment solution; dental rinse tent solution; ingestible blood oxygen content elevating solutions; ingestible blood oxygen partial pressure elevating solutions; bactericide; virus killing solution; anaerobic tumour treatment solution; physical injury immersion treatment solution; and brain tissue treatment solution.

28. The use of super-oxygenated water as defined in claim 23 for the manufacture of a preservative selected from the group consisting of: fluid preservative; ice used as a cooling preservative; live human organ preservative; and live human tissue preservative.
